(19) United States
(12) Patent Application Publication
(10) Pub. No.: US 2003/0219405 A1
(43) Pub. Date: Nov. 27, 2003

(54) Oral administration of interferon-tau

(76) Inventors: Yoshihiro Sokawa, Kyoto (JP); Chih-Ping Liu, San Francisco, CA (US)

Correspondence Address: PERKINS COIE LLP, P.O. BOX 2168, MENLO PARK, CA, 94026, US

(21) Appl. No.: 10/346,269

(22) Filed: Jan. 16, 2003

Related U.S. Application Data
(60) Provisional application No. 60/349,658, filed on Jan. 16, 2002.

(51) Int. Cl. A61K 38/21

(52) U.S. Cl. 424/85.4

(57) ABSTRACT

A method of administering interferon-τ to a subject subsequent to a defined food and/or water intake regimen is described. The method comprises administering orally to the subject, subsequent to fasting and/or fasting combined with a controlled or absence of fluid intake, an amount of interferon-τ that is effective to achieve an increased level of 2′,5′-oligoadenylate synthetase (OAS) activity in whole blood relative to that achieved from oral administration to a subject also treated with interferon-τ but not held to the defined food and/or water intake regimen.

### CROSS REFERENCE TO RELATED APPLICATIONS

This application claims the benefit of U.S. Provisional Application No. 60/349,658, filed Jan. 16, 2002, incorporated herein by reference in its entirety.

### FIELD OF THE INVENTION

The present invention relates generally to oral delivery of cytokines and more particularly to oral delivery of interferons.

### BACKGROUND

In recent years, the variety of therapeutic agents for treatment of physiological conditions and disease states has expanded considerably, due in large part to the growing use of polypeptides and proteins as therapeutic agents. The important role of peptides in replacement therapy and as pharmaceutical agents is reflected in the efforts toward synthesis of large quantities of proteins by recombinant DNA technology.

One limiting factor in the use of proteins and polypeptides as therapeutic agents is metabolization by plasma proteins when given parenterally. The oral route of administration is even more problematic due to proteolysis in the stomach, where the acidic conditions can destroy the molecule before reaching its intended target. For example, polypeptides and protein fragments, produced by action of gastric and pancreatic enzymes, are cleaved by exo- and endopeptidases in the intestinal brush border membrane to yield di- and tri-peptides. If proteolysis by pancreatic enzymes is avoided, polypeptides are subject to degradation by brush border peptidases. Polypeptides or proteins that might survive passage through the stomach are subject to metabolism in the intestinal mucosa where a penetration barrier prevents entry into cells.

Despite these obstacles, therapeutically beneficial oral delivery of proteins and polypeptides can be achieved, typically by formulating the molecule in a protective dosage form for survival in the stomach and intestines until absorbed by the intestinal mucosa. For example, the protein can be co-administered with protease inhibitors, stabilized with polymeric materials, or encapsulated in a lipid or polymer particle. Another approach is to avoid the gastrointestinal tract altogether, by delivering the protein to the oral-pharyngeal region in the form of a lozenge or solution held in the oral cavity for a period of time.

Another factor that must be considered in oral administration of compounds are food-drug interactions that may alter the pharmacokinetics and pharmacodynamic profile of the orally administered drug. Food effects on drug absorption and bioavailability have been studied for small drug molecules (see for example, Singh, B., Clin. Pharmacokinet, 37(3):213 (1999)), but less is known about food effects on absorption and bioavailability of proteins and peptides and it is not clear that the mechanisms for smaller drug compounds apply to proteins and peptides. Even for small drug compounds, it is unknown a priori what effect stomach contents will have on the compound. There are typically five categories of food effects on absorption of small drug molecules: those causing (1) decreased (2) delayed; (3) increased; or (4) accelerated absorption, and (5) those in which food has no significant effect. There are a number of variables that interface between differential effects of food and postprandial bioavailability are (i) the physicochemical characteristics and composition of the drug; (ii) timing of the meal in relation to time of drug administration; (iii) size and composition of meals; and (iv) dosage. Further, the mechanism of “food effect” may involve physiological and sensory responses to food, such as changes in gastro-intestinal milieu and gastric emptying rate, and reflux action (Id.)

Although there is a vast amount of literature on food effects on small drug compounds, there is still no basis to predict the effect of food for a particular chemical entity or a chemical class of therapeutic agents (Id.). Moreover, there is no basis for knowing if the studies on small drug compounds are applicable to proteins and polypeptides; and even if they are, there is simply no way to know what effect food and/or water intake will have on an orally administered non-native protein like interferon-tau.

Interferon-tau (hereinafter “IFN-τ” or interferon-τ) was discovered originally as a pregnancy recognition hormone produced by the trophectoderm of ruminant conceptuses (Imakawa, K., et al, Nature 330:377-379, (1987); Bazer, F. W. and Johnson, H. M., Am J Repro Immunol 26:19-22, (1991)). Although the distribution of the IFN-τ gene is restricted to ruminants including cattle, sheep, and goats, (Alexenko, A. P., et al., J Interferon and Cytokine Res 19:1335-1341, (1999)) IFN-τ exhibits activity in cells belonging to other species including humans and mice (Pontzer, C. H., et al., Cancer Res 51:5304-5307, (1991); Alexenko, A. P., et al., J Interferon and Cytokine Res 20:817-822, (2000)). For example, IFN-τ has been demonstrated to possess antiviral, (Pontzer, C. H., et al., Biochem Biophys Res Commun 152:801-807, (1988)), antiprolipherative, (Pontzer, C. H., et al., 1991) and immunoregulatory activities (Assal-Meliani, A., Am J Repro Immunol 33:267-275, (1995)).

While IFN-τ displays many of the activities classically associated with type I IFNs, such as interferon-α and inteferon-β, considerable differences exist between IFN-τ and the other type I IFNs. The most prominent difference is the role of IFN-τ in pregnancy in ruminant species. The other IFNs have no similar activity in pregnancy recognition. Also different is viral induction. All type I IFNs, except IFN-τ, are induced readily by virus and dsRNA (Roberts, et al., Endocrine Reviews 13:432 (1992)). Induced IFN-α and IFN-β expression is transient, lasting approximately a few hours. In contrast, IFN-τ synthesis, once induced, is maintained over a period of days (Godkin, et al., J. Reprod Fert. 65:141 (1982)). On a per-cell basis, 300-fold more IFN-τ is produced than other type I IFNs (Cross, J. C. and Roberts, R. M., Proc. Natl. Acad. Sci. USA 88:3817-3821 (1991)).

Another difference lies in the amino acid sequences of IFN-τ and other type I interferons. The percent amino acid sequence similarity between the interferons α_{2b}, β₁, ω₁, γ, and τ are summarized in the table below.

| | rHuIFNα_{2b} | rHuIFNβ₁ | rHuIFN₁ω₁ | rHuIFN_{γ} | rOvIFNτ |
|---|---|---|---|---|---|
| RhuIFNα_{2b} | | 33.1 | 60.8 | 11.6 | 48.8 |
| RhuIFNβ₁ | 33.1 | | 33.1 | 12.2 | 33.8 |
| RhuIFNω₁ | 60.8 | 33.1 | | 10.2 | 54.9 |
| RhuIFN_{γ} | 11.6 | 12.2 | 10.2 | | 10.2 |
| roIFNτ | 48.8 | 33.8 | 54.9 | 10.2 | |

Sequence comparison determined from the following references:
Taniguchi et al., Gene, 10(1):11 (1980).
Adolf et al., Biochim. Biophys. Acta, 1089(2):167 (1991).
Streuli et al., Science, 209:1343 (1980).
Imakawa et al., Nature, 330:377 (1987).

Recombinant ovine IFNτ (rOvIFNτ) is 48.8 percent homologous to IFNα_{2b} and 33.8 percent homologous to IFNβ₁. Because of this limited homology between IFNτ and IFNα and between IFNτ and IFNβ, it cannot be predicted whether or not IFNτ would behave in the same manner as IFNα or IFNβ when administered orally. Teachings in the art relating to oral administration of IFNα, IFNβ, or any other non-tau interferon, fail to provide a basis for drawing any expectations for IFN-τ.

### SUMMARY OF THE INVENTION

Accordingly, in one aspect the invention includes a method of administering interferon-τ to a subject subsequent to a defined food and/or water intake regimen. The method comprises administering orally to the subject an amount of interferon-τ that is effective to achieve an increased level of 2′,5′-oligoadenylate synthetase (OAS) activity in whole blood relative to that achieved from oral administration to a subject also treated with interferon-τ but not held to a defined food and/or water intake regimen.

In one embodiment, the interferon-τ is ovine or bovine interferon-τ.

The interferon-τ can be administered in the form of a solid dosage form or as a liquid dosage form. An exemplary dosage is of at least about 1×10⁴ Units/day.

In another aspect, the invention contemplates a method administering interferon-τ, comprising (i) withholding food from a subject selected for administration of interferon-τ; and (ii) orally administering interferon-τ to the subject to achieve an increased level of 2′,5′-oligoadenylate synthetase in the blood relative to the level of 2′,5′-oligoadenylate synthetase in the blood obtained after oral administration of interferon-τ to a fed subject.

In one embodiment, withholding further includes withholding water from the subject. In another embodiment, withholding comprises withholding food from the subject for at least one hour, more preferably, for at least four hours, still more preferably for at least six hours, prior to oral administration.

The method of the invention, in another embodiment, finds use in treating an autoimmune condition, a viral infection, or a condition associated with cellular proliferation.

In yet another aspect, an improvement in a method of oral administration of interferon-τ is contemplated. The improvement comprises withholding food from a subject prior to oral administration of IFN-τ to the subject. Such withholding is effective to achieve an increased level of 2′,5′-oligoadenylate synthetase in the blood relative to the level of 2′,5′-oligoadenylate synthetase in the blood obtained after oral administration of interferon-τ to a fed subject.

These and other objects and features of the invention will become more fully apparent when the following detailed description is read in conjunction with the accompanying drawings.

### BRIEF DESCRIPTION OF THE DRAWINGS

FIGS. 1A-1D are bar graphs showing the effect of fasting conditions on the induction of blood OAS in mice by administration of OvIFN-τ. The induction of blood OAS is shown as a percentage of control, taken as blood OAS in mice treated with a solution of 10% maltose without interferon. Treated mice received 10⁴ U of OvINFτ (via intraperitoneal injection or oral administration) six hours after the indicated intake regimen: FIG. 1A, no food and no water; FIG. 1B, water without food; FIG. 1C, food without water; FIG. 1D, both food and water. Each bar represents the average±S.E. of one experiment (3 mice) of two performed, with similar results.

FIG. 2 is a bar graph showing blood OAS concentration, in pmol/dL, in several mouse strains (ICR, BALB/c, C57BL, NZW/N and SJL/J) following peroral administration of OvIFN-τ (10⁵ U). Control mice received orally a solution of 10% maltose without IFN. Each bar represents the average±S.E. of one experiment (3˜5 mice) of two performed, with similar results.

FIGS. 3A-3B are bar graphs showing induction of blood OAS activity in mice after administration of OvIFNτ following a 6 hour fast. IFNτ was administered orally or via intraperitoneal injection. FIG. 3A shows blood OAS levels, expressed as a percentage of control (see description in FIG. 1 above) in blood samples taken at time zero and at 8 hours, 16 hours, and 24 hours post IFNτ (10⁵ U) administration. FIG. 3B shows blood OAS levels, expressed as a percentage of control, 24 hours after delivery of OvIFNτ at concentrations of 0, 10², 10³, 10⁴, and 10⁵ U. Each bar represents the average±S.E. of one experiment (3 mice) of two performed, with similar results.

FIG. 4 is a bar graph showing induction of blood OAS activity, expressed as a percentage of control (see description of control in FIG. 1 above), by administration of MuIFNα (0, 10², 10³ and 10⁴ IU) given to ICR mice by orally or via intraperitoneal injection. The OAS activity in blood was assayed 16 hours after IFNα administration. Each bar represents the average±S.E. of one experiment (3 mice) of two performed, with similar results.

### BRIEF DESCRIPTION OF THE SEQUENCES

SEQ ID NO:1 is the nucleotide sequence of a synthetic gene encoding ovine interferon-τ. Also shown is the encoded amino acid sequence.

SEQ ID NO:2 is an amino acid sequence of a mature OvIFN-τ protein.

### DETAILED DESCRIPTION OF INVENTION

I. Definitions

“Fasted state” or “fasting conditions” intend abstaining from all food and drinking only water for at least about one hour, preferably for at least about two hours, more preferably for at least about four hours, most preferably for at least about six hours, prior to oral administration of a therapeutic agent, such as a protein or peptide.

“Fasted state also excluding water” intends abstaining from all food and all fluids, including but not limited to water, for at least about one hour, preferably for at least about two hours, more preferably for at least about four hours, most preferably for at least about six hours, prior to oral administration of a therapeutic agent, such as a protein or peptide.

“Non-fasted state” or “fed state” intend consumption of food and/or water at any time prior to oral administration of a therapeutic agent, such as a protein or peptide.

“Withholding food” intends a fasted state.

“Orally administering” or “oral administration” intend delivery of a compound to the stomach and/or gastro-intestinal system of a subject. These terms do not include oral-pharyngeal delivery, where systemic delivery of a compound is achieved by absorption in the oral cavity or pharyngeal area.

“Peptide” and “polypeptide” are used interchangeably herein and refer to a compound made up of a chain of amino acid residues linked by peptide bonds. Unless otherwise indicated, the sequence for peptides is given in the order from the amino terminus to the carboxyl terminus.

When a first peptide or polypeptide is said to “correspond” or to be “homologous” to a second peptide or polypeptide fragment, it means that the peptide or fragments have a similarity in amino acid residues if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp. 1-10.) The two sequences (or parts thereof) are more preferably homologous if their amino acids are greater than or equal to 50%, more preferably 70%, still more preferably 80%, identical when optimally aligned using the ALIGN program mentioned above.

A polypeptide sequence or fragment is “derived” from another polypeptide sequence or fragment when it has an identical sequence of amino acid residues as a region of the another sequence or fragment.

An interferon-τ polypeptide is a polypeptide having between about 15 and 172 amino acids derived from an interferon-τ amino acid coding sequence, where said 15 to 172 amino acids are contiguous in native interferon-τ. Such 15-172 amino acid regions can also be assembled into polypeptides where two or more such interferon-τ regions are joined that are normally discontinuous in the native protein.

Treating a disease refers to administering a therapeutic substance effective to reduce the symptoms of the disease and/or lessen the severity of the disease.

II. Method of Interferon Administration

A. Interferon-τ

The 172 amino acid sequence of ovine-IFNτ is set forth, for example, in U.S. Pat. No. 5,958,402, and is also set forth herein as SEQ ID NO:2. IFNτ sequences with similar characteristics and activities to ovine IFN-τ have been isolated from other ruminant species including cows and goats (Bartol, F. F., et al., Biol. Reprod. 32:681-693, (1985); Gnatek, G. G., et al., Biol. Reprod. 41:655-664, (1989); Helmer, S. D., et al., J. Reprod. Fert. 79:83-91, (1987); and Imakawa, K., et al., Mol. Endocrinol. 3:127, (1989)). Bovine IFNτ (BoIFNτ) and OvIFNτ (i) have similar functions in maternal recognition of pregnancy, and (ii) share a high degree of amino acid and nucleotide sequence homology between mature proteins. The nucleic acid sequence homology between OvIFNτ and BoIFNτ is 76.3% for the 5′ non-coding region, 89.7% for the coding region, and 91.9% for the 3′ non-coding region. The amino acid sequence homology is 80.4%. The homologous bovine-IFNτ sequence is described, for example, in Helmer et al., J. Reprod. Fert. 79:83-91, (1987) and Imakawa, K. et al., Mol. Endocrinol. 3:127, (1989). The sequences of ovine-IFNτ and bovine-IFNτ from these references are hereby incorporated by reference.

B. Method of Administration

In studies performed in support of the invention, OvIFN-τ was administered orally to mice and the induction of 2′,5′-oligoadenylate synthetase (OAS) activity, a recognized marker of IFN action (Shindo, M., et al., Hepatology 8:366-370, (1988)), in whole blood was monitored. In all of the studies describe below, the procedure set forth in Example 1 was followed. Before administration of OvIFN-τ, mice were deprived of food and drink for at least six hours and IFN-τ was given by peroral (p.o.) administration and, for a comparative control, by intraperitoneal (i.p.) injection. When administered orally, IFN-τ was introduced directly into the upper part of the stomach using an oral feeding needle.

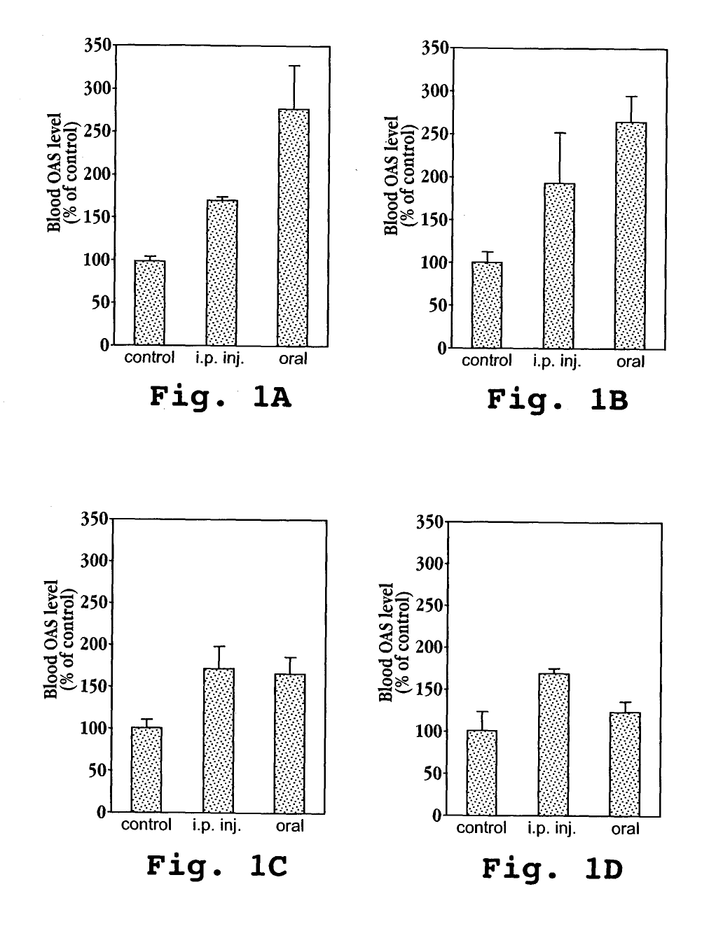

In an initial study, the effect of fasting conditions on the induction of blood OAS in mice by administration of OvIFN-τ was evaluated. In this study, mice were subjected to a defined food and water intake regimen for six hours. After the six hour regimen, 10⁴ U of OvINFτ was administered by oral gavage or by intraperitoneal injection, along with food and water. The intake regimens were as follows: Case I, neither food nor water was given; Case II, water but no food was given; Case III, only food was given; Case IV, both food and water were given. Whole blood was obtained from the heart at 24 hours and levels of OAS activity were determined. The results are shown in FIGS. 1A-1D.

FIGS. 1A-1D correspond to the mice subjected to Case I-Case IV food and water intake regimens defined in the paragraph above, respectively. The results in FIGS. 1A-1D are show the induction of blood OAS expressed as a percentage of control, taken as blood OAS in mice treated with a solution of 10% maltose without interferon. The results show that higher blood OAS levels are induced by oral administration of IFN-τ to subjects in a fasted state, as seen best in FIG. 1A and FIG. 1B for mice receiving no food.

In this study, it was observed that almost the same amounts of food were ingested with or without a supply of water. Water intake, however, was lower without food (case I and case II) than with food (case III and case IV). In some animals, after fasting for six hours, a 0.2-ml maltose solution containing blue dye was given orally and the distribution of the dye in the stomach and intestine was examined (data not shown). Following the ingestion of food (case III and case IV), the stomachs of mice swelled and the dye localized mainly in the stomach, probably because the food absorbed the dye. However, the dye was transferred quickly to the intestine when no food was ingested. This observation suggests that OvIFN-τ taken orally may exert its effect in the intestine to induce high levels of OAS activity in blood.

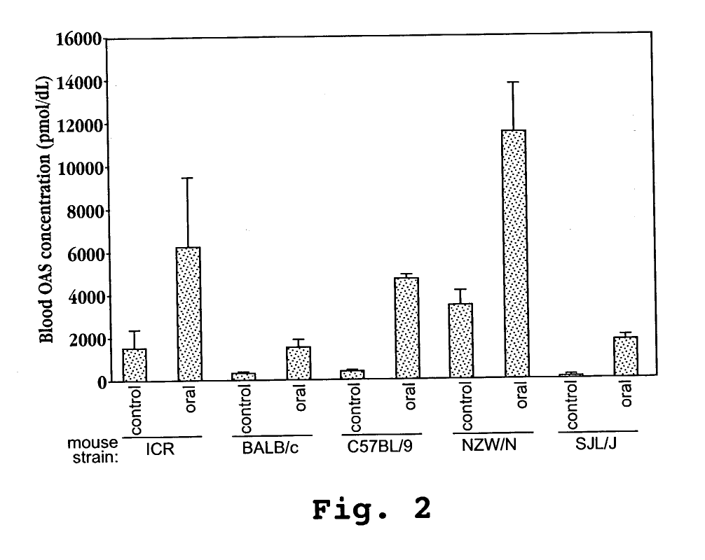

FIG. 2 shows the effects of gastric administration of OvIFN-τ on the induction of OAS activity in blood in a variety of mouse strains: ICR, BALB/c, C57BL/9, NZW/N and SJL/J. All test mice were treated orally with OvIFN-τ (10⁵ U). Control mice received orally a solution of 10% maltose without IFN-τ. Each bar represents the average±S.E. of one experiment (3˜5 mice) of two performed, with similar results.

As seen in FIG. 2, the level of OAS activity in all mouse strains increased following peroral administration of OvIFN-τ, though the extent of the increase varied with the strain. The level of activity induced in ICR, C57BL/9 and NZW/N mice was higher than that in BALB/c and SJL/J mice.

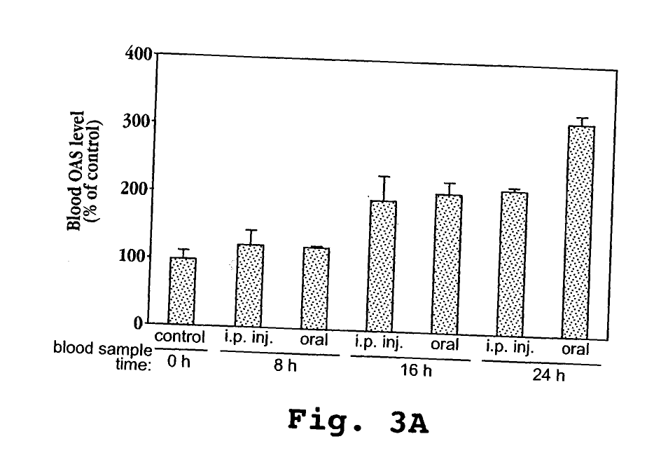

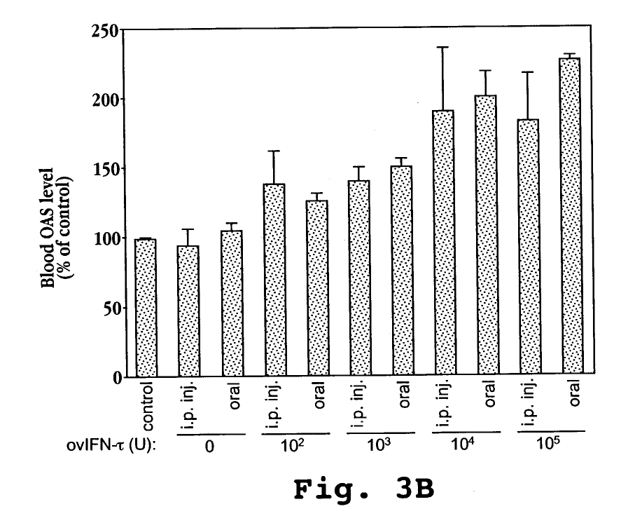

In another study, OAS activity was monitored as a function of time after administration of IFN-τ. In this study, animals (ICR mice) were subjected to a six hour fast (water but no food) prior to administration of IFN-τ (10⁵U). Blood was sampled at 8 hours, 16 hours, and 24 hours post IFN-τ administration. The results are shown in FIG. 3A.

FIG. 3A shows blood OAS levels, expressed as a percentage of control (see description in FIG. 1 above), in blood samples taken at the indicated time intervals post IFNτ administration. In FIG. 3A, each bar represents the average±S.E. of one experiment (3 mice) of two performed, with similar results. OAS activity in whole blood increased in a time-dependent manner regardless of the route, oral or i.p. injection, however, a higher level was observed at the 24 hour time point after oral administration than i.p. injection.

In another study, OvIFN-τ at varying concentrations (0, 10², 10³, 10⁴ and 10⁵ U), was given to mice following a six hour fast. Blood was obtained after 24 hours, and OAS activity was assayed. The results are shown in FIG. 3B.

FIG. 3B shows blood OAS levels, expressed as a percentage of control (see control description in FIG. 1 above), 24 hours after delivery of OvIFNτ at concentrations of 0, 10², 10³, 10⁴, and 10⁵ U. Each bar represents the average±S.E. of one experiment (3 mice) of two performed, with similar results. Following i.p. injection, the level of activity was rather high at a low dose (10² U), and saturated at higher doses of OvIFN-τ (10⁴ and 10⁵ U). In contrast, the level of activity after p.o. administration increased dose-dependently.

The data in FIGS. 3A-3B shows that IFN-τ administered orally induces a higher level of blood OAS activity than that induced by i.p. injection. In particular, the orally-induced blood OAS levels were higher than the blood OAS levels induced by i.p. injection at IFN-τ dosages of greater than about 10³ U and at post administration times of greater than about 8 hours.

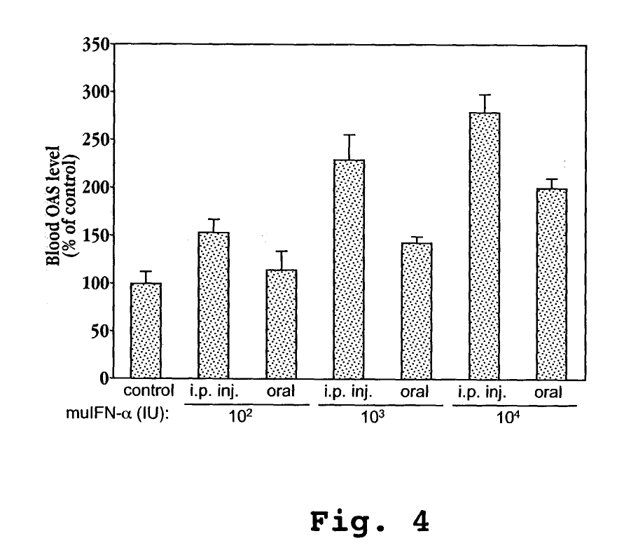

A comparative study was done to measure the effect of oral administration of MuIFN-α on blood OAS levels. In this study, ICR mice were treated with varying concentrations (0, 10², 10³ and 10⁴ IU) of MuIFN-α by either the p.o. or i.p. route. OAS activity in blood obtained 16 hours after MuIFN-α administration was assayed. The results are shown in FIG. 4, where each bar represents the average±S.E. of one experiment (3 mice) of two performed, with similar results.

FIG. 4 is a bar graph showing induction of blood OAS activity, expressed as a percentage of control (see description of control in FIG. 1 above), following administration of MuIFNα (0, 10², 10³ and 10⁴ IU) given orally or via intraperitoneal injection. The level of OAS activity was increased dose-dependently by either route of administration, with i.p. injection resulting in better induction of blood OAS activity than p.o. administration. This result is the opposite of that observed with IFN-τ, where oral administration of IFN-τ achieved a higher blood OAS level than intraperitoneal injection of IFN-τ. Moreover, the body temperature of mice rose slightly when MuIFN-α was administered, but not when OvIFN-τ was used (data not shown).

III. Utility

A. Treatment of Conditions Responsive to IFN-τ

As noted above, IFN-τ has biological activity as an antiviral agent, an anti-proliferative agent, and in treatment of autoimmune disorders (see for example U.S. Pat. Nos. 5,958,402; 5,942,223; 6,060,450; 6,372,206, which are incorporated by reference herein). Accordingly, the invention contemplates oral administration of IFN-τ for treatment of any condition responsive to IFN-τ when administered via injection. Conditions and diseases which may be treated using methods of the present invention include autoimmune, inflammatory, proliferative and hyperproliferative diseases, as well as immunologically-mediated diseases.

In particular, methods of the present invention are advantageous for treating conditions relating to immune system hypersensitivity. There are four types of immune system hypersensitivity (Clayman, C. B., Ed., American Medical Association Encyclopedia of Medicine, Random House, New York, N.Y., (1991)). Type I, or immediate/anaphylactic hypersensitivity, is due to mast cell degranulation in response to an allergen (e.g., pollen), and includes asthma, allergic rhinitis (hay fever), urticaria (hives), anaphylactic shock, and other illnesses of an allergic nature. Type II, or autoimmune hypersensitivity, is due to antibodies that are directed against perceived “antigens” on the body's own cells. Type III hypersensitivity is due to the formation of antigen/antibody immune complexes which lodge in various tissues and activate further immune responses, and is responsible for conditions such as serum sickness, allergic alveolitis, and the large swellings that sometimes form after booster vaccinations. Type IV hypersensitivity is due to the release of lymphokines from sensitized T-cells, which results in an inflammatory reaction. Examples include contact dermatitis, the rash of measles, and “allergic” reactions to certain drugs.

The mechanisms by which certain conditions may result in hypersensitivity in some individuals are generally not well understood, but may involve both genetic and extrinsic factors. For example, bacteria, viruses or drugs may play a role in triggering an autoimmune response in an individual who already has a genetic predisposition to the autoimmune disorder. It has been suggested that the incidence of some types of hypersensitivity may be correlated with others. For example, it has been proposed that individuals with certain common allergies are more susceptible to autoimmune disorders.

Autoimmune disorders may be loosely grouped into those primarily restricted to specific organs or tissues and those that affect the entire body. Examples of organ-specific disorders (with the organ affected) include multiple sclerosis (myelin coating on nerve processes), type I diabetes mellitus (pancreas), Hashimotos thyroiditis (thyroid gland), pernicious anemia (stomach), Addison's disease (adrenal glands), myasthenia gravis (acetylcholine receptors at neuromuscular junction), rheumatoid arthritis (joint lining), uveitis (eye), psoriasis (skin), Guillain-Barré Syndrome (nerve cells) and Grave's disease (thyroid). Systemic autoimmune diseases include systemic lupus erythematosus and dermatomyositis.

Other examples of hypersensitivity disorders include asthma, eczema, atopical dermatitis, contact dermatitis, other eczematous dermatitides, seborrheic dermatitis, rhinitis, Lichen planus, Pemplugus, bullous Pemphigoid, Epidermolysis bullosa, uritcaris, angioedemas, vasculitides, erythemas, cutaneous eosinophilias, Alopecia areata, atherosclerosis, primary biliary cirrhosis and nephrotic syndrome. Related diseases include intestinal inflammations, such as Coeliac disease, proctitis, eosinophilia gastroenteritis, mastocytosis, inflammatory bowel disease, Crohn's disease and ulcerative colitis, as well as food-related allergies.

Autoimmune diseases particularly amenable for treatment using the methods of the present invention include multiple sclerosis, type I (insulin dependent) diabetes mellitus, lupus erythematosus, amyotrophic lateral sclerosis, Crohn's disease, rheumatoid arthritis, stomatitis, asthma, uveitis, allergies and psoriasis.

Methods of the present invention may be used to therapeutically treat and thereby alleviate autoimmune disorders such as those discussed above.

In another embodiment, the methods of the invention are used to treat conditions associated with viral infection. The antiviral activity of IFN-τ has broad therapeutic applications without the toxic effects that are usually associated with IFNαs, and IFN-τ exerts its therapeutic activity without adverse effects on the cells. The relative lack of cytotoxicity of IFN-τ makes it extremely valuable as an in vivo therapeutic agent and sets IFN-τ apart from most other known antiviral agents and all other known interferons.

Formulations containing IFN-τ can be orally-administered to inhibit viral replication. Examples of specific viral diseases which may be treated by orally-administered IFNτ include, but are not limited to, hepatitis A, hepatitis B, hepatitis C, non-A, non-B, non-C hepatitis, Epstein-Barr viral infection, HIV infection, herpes virus (EB, CML, herpes simplex), papilloma, poxvirus, picorna virus, adeno virus, rhino virus, HTLV I, HTLV II, and human rotavirus.

In another embodiment, the methods of the invention are contemplated for treatment of conditions characterized by hyperproliferation. IFN-τ exhibits potent anticellular proliferation activity. Accordingly, a method of inhibiting cellular growth by orally administering IFN-τ is contemplated, in order to inhibit, prevent, or slow uncontrolled cell growth.

Examples of specific cell proliferation disorders which may be treated by orally-administered IFN-τ include, but are not limited to, hairy cell leukemia, Kaposi's Sarcoma, chronic myelogenous leukemia, multiple myeloma, superficial bladder cancer, skin cancer (basal cell carcinoma and malignant melanoma), renal cell carcinoma, ovarian cancer, low grade lymphocytic and cutaneous T cell lymphoma, and glioma.

In addition to the uses of the methods of the present invention detailed above, it will be appreciated that the methods may be applied to the treatment of a variety of immune system disorders suffered by domesticated and wild animals. For example, hypothyroidism in dogs typically results from a progressive destruction of the thyroid, which may be associated with lymphocytic thyroiditis (Kemppainen, R. J., and Clark, T. P., Vet Clin N Am Small Anim Pract 24(3):467-476, (1994)). Lymphocytic thyroiditis, which resembles Hashimoto's thyroiditis in humans, is thought to be an autoimmune disorder. According to the guidance presented herein, hypothyroidism due to lymphocytic thyroiditis in dogs may be treated with IFN-τ as described above.

Another type of autoimmune disorder in dogs that may be alleviated by treatment with IFN-τ is characterized by antinuclear antibody (ANA) positivity, pyrexia and seronegative arthritis (Day, M. J., et al., Clin Immunol Immunopathol 35(1):85-91,(1985)). Immune-mediated thrombocytopenia (ITP; Kristensen, A. T., et al., J Vet Intern Med 8(1):36-39, (1994); Werner, L. L., et al., Vet Immunol Immunopathol 8(1-2):183-192, (1985)), systemic lupus erythematosus (Kristensen, et al., 1994), and leukopenia and Coomb's positive hemolytic anemia (Werner, et al., 1985), may also be amenable to treatment using methods of the present invention.

B. Formulations and Dosages

Oral preparations containing IFN-τ can be formulated according to known methods for preparing pharmaceutical compositions. In general, the IFN-τ therapeutic compositions are formulated such that an effective amount of the IFN-τ is combined with a suitable additive, carrier and/or excipient in order to facilitate effective oral administration of the composition. For example, tablets and capsules containing IFN-τ may be prepared by combining IFN-τ (e.g., lyophilized IFN-τ protein) with additives such as pharmaceutically acceptable carriers (e.g., lactose, corn starch, microcrystalline cellulose, sucrose), binders (e.g., alpha-form starch, methylcellulose, carboxymethylcellulose, hydroxypropylcellulose, hydroxypropylmethylcellulose, polyvinylpyrrolidone), disintegrating agents (e.g., carboxymethylcellulose calcium, starch, low substituted hydroxy-propylcellulose), surfactants (e.g., Tween 80, polyoxyethylene-polyoxypropylene copolymer), antioxidants (e.g., L-cysteine, sodium sulfite, sodium ascorbate), lubricants (e.g., magnesium stearate, talc), or the like.

Further, IFN-τ polypeptides of the present invention can be mixed with a solid, pulverulent or other carrier, for example lactose, saccharose, sorbitol, mannitol, starch, such as potato starch, corn starch, millopectine, cellulose derivative or gelatine, and may also include lubricants, such as magnesium or calcium stearate, or polyethylene glycol waxes compressed to the formation of tablets. By using several layers of the carrier or diluent, tablets operating with slow release can be prepared.

Liquid preparations for oral administration can be made in the form of elixirs, syrups or suspensions, for example solutions containing from about 0.1% to about 30% by weight of IFN-τ, sugar and a mixture of ethanol, water, glycerol, propylene, glycol and possibly other additives of a conventional nature.

An orally-active IFN-τ pharmaceutical composition is administered in a therapeutically-effective amount to an individual in need of treatment. The dose may vary considerably and is dependent on factors such as the seriousness of the disorder, the age and the weight of the patient, other medications that the patient may be taking and the like. This amount or dosage is typically determined by the attending physician. The dosage will typically be between about 1×10⁴ and 1×10⁹ units/day, more preferably between 1×10⁵ and 1×10⁸ units/day, preferably between about 1×10⁶ and 1×10⁷ units/day. In one specific embodiment, IFN-τ is administered orally at a dosage of greater than about 1×10⁴ units/day, preferably of greater than about 1×10⁶ units/day, more preferably greater than about 1×10⁸ units/day.

Disorders requiring a steady elevated level of IFN-τ in plasma will benefit from administration as often as about every two to four hours, while other disorders, such as multiple sclerosis, may be effectively treated by administering a therapeutically-effective dose at less frequent intervals, e.g., once every 48 hours. The rate of administration of individual doses is typically adjusted by an attending physician to enable administration of the lowest total dosage while alleviating the severity of the disease being treated.

Once improvement of a patient's condition has occurred, a maintenance dose is administered if necessary. Subsequently, the dosage or the frequency of administration, or both, may be reduced, as a function of the symptoms, to a level at which the improved condition is retained.

As noted above, oral administration of IFN-τ is prescribed along with a defined food/water intake regimen. It will be appreciated that the food/water intake regimen selected for the supporting studies described with respect to FIGS. 1A-3B are merely exemplary. The invention contemplated that food and/or water can be withheld for a variety of times prior to dosing with the protein, ranging from more or less than the 6 hours exemplified here. In a preferred embodiment, food and/or water are withheld for at least about one hour prior to oral administration of IFN-τ, more preferably are withheld for at least about two hours, still more preferably for at least about six hours.

It will, of course, be understood that the oral administration of IFN-τ in accord with the invention may be used in combination with other therapies. For example, IFN-τ can be accompanied by administration of an antigen against which an autoimmune response is directed. Examples include co-administration of myelin basic protein and IFN-τ to treat multiple sclerosis; collagen and IFN-τ to treat rheumatoid arthritis, and acetylcholine receptor polypeptides and IFN-τ to treat myasthenia gravis.

Furthermore, IFN-τ may be orally administered with known immunosuppressants, such as steroids, to treat autoimmune diseases such as multiple sclerosis. The immunosuppressants may act synergistically with IFN-τ and result in a more effective treatment that could be obtained with an equivalent dose of IFN-τ or the immunosuppressant alone.

Similarly, in a treatment for a cancer or viral disease, IFN-τ may be administered in conjunction with, e.g., a therapeutically effective amount of one or more chemotherapy agents such as busulfan, 5-fluorouracil (5-FU), zidovudine (AZT), leucovorin, melphalan, prednisone, cyclophosphamide, dacarbazine, cisplatin, dipyridamole, and the like.

### IV. EXAMPLES

The following example illustrates but in no way is intended to limit the present invention.

### Example 1

Method of Oral Administration

Pathogen-free 5-week-old female mice of the ICR, BALB/c, C57BL/9, NZW/N and SJL/J strains were purchased from Japan SLC. Inc., Hamamatsu. The mice were reared one week in the laboratory before experiments.

Recombinant ovine IFN-τ (OvIFN-τ) was obtained from Pepgen Corporation (Alameda, Calif.). The IFN belongs to the subtype of OvIFN-τ1. The preparation used in this study had a specific activity of 5×10⁸ units (U)/mg protein as assayed in MDBK cells challenged with VSV and standardized against human IFN-α. Natural murine IFN-α (MuIFN-α) was supplied by Sumitomo Pharmaceutical Co. (Osaka, Japan), whose specific activity was 1×10⁸ international units (IU)/mg protein.

For administration to the mice, IFN-τ was dissolved in a solution containing 10% maltose. Samples of 0.2 ml were administered to mice (6-week-old females) by either peroral (p.o.) treatment or intraperitoneal (i.p.) injection. When given orally, the samples were introduced directly into the upper part of the stomach using a 20 gauge oral feeding needle. Before the administration, mice were deprived of both food and drink for 6 hours, starting at 1 pm and ending at 7 pm. After the fasting, IFN was administered by either the p.o. or i.p. route and food and drink were given at 6 hours. Then, whole blood was obtained from the heart at 24 hours.

The 2′,5′-oligoadenylate synthetase (OAS) activity in whole blood was assayed with Eiken's 2-5A RIA kit. Diluted blood was mixed with polyl:C-agarose gel, ATP was added after washing the gel, and the 2-5A produced was assayed by the RIA method (Shindo, M., et al., 1988). The assays were performed twice in each sample. For the estimation of the level of blood OAS, at least three mice were used.

It is claimed:

1. A method of administering interferon-tau (interferon-τ), comprising
orally administering interferon-τ to a patient in a fasted state, whereby said administering is effective to achieve an increased level of 2′,5′-oligoadenylate synthetase in the blood relative to the level of 2′,5′-oligoadenylate synthetase in the blood obtained after oral administration of interferon-τ to a patient in a non-fasted state.

2. The method of claim 1, wherein said interferon-τ is ovine or bovine interferon-τ.

3. The method of claim 2, wherein said interferon-τ has a sequence corresponding to the amino acid sequence presented as SEQ ID NO:2.

4. The method of claim 1, wherein said orally administering is by oral administration of a solid dosage form.

5. The method of claim 1, wherein said orally administering is by oral administration of a liquid dosage form.

6. The method of claim 1, wherein said orally administering is at a dose of at least about 1×10⁴ Units/day.

7. A method of administering interferon-τ, comprising
withholding food from a subject selected for administration of interferon-τ; and
orally administering interferon-τ to the subject, whereby said administering is effective to achieve an increased level of 2′,5′-oligoadenylate synthetase in the blood relative to the level of 2′,5′-oligoadenylate synthetase in the blood obtained after oral administration of interferon-τ to a fed subject.

8. The method of claim 7, wherein said withholding further includes withholding water from said subject.

9. The method of claim 7, wherein said withholding comprises withholding food from said subject for at least one hour prior to oral administration.

10. The method of claim 7, wherein said withholding comprises withholding food and water from said subject for at least four hours prior to oral administration.

11. The method of claim 7, wherein said withholding comprises withholding food from said subject for at least six hours prior to oral administration.

12. The method of claim 7, wherein said interferon-τ is ovine or bovine interferon-τ.

13. The method of claim 12, wherein said interferon-τ has an amino acid sequence corresponding to the sequence presented as SEQ ID NO:2.

14. The method of claim 7, wherein said orally administering includes orally administering interferon-τ as a solid dosage form.

15. The method of claim 7, wherein said orally administering includes orally administering interferon-τ as a liquid dosage form.

16. The method of claim 7, wherein said orally administering is at a dose of at least about 1×10⁴ Units/day.

17. The method of claim 7, wherein said administering is effective to treat an autoimmune condition.

18. An improvement in a method of oral administration of interferon-τ, comprising
prior to oral administration of IFN-τ to a subject, withholding food from the subject, whereby said withholding is effective to achieve an increased level of 2′,5′-oligoadenylate synthetase in the blood relative to the level of 2′,5′-oligoadenylate synthetase in the blood obtained after oral administration of interferon-τ to a fed subject.

19. The method of claim 18, wherein said withholding further includes withholding water from said subject.

20. The method of claim 18, wherein said withholding comprises withholding food from said subject for at least one hour prior to oral administration.

21. The method of claim 18, wherein said withholding comprises withholding food and water from said subject for at least four hours prior to oral administration.

22. The method of claim 18, wherein said withholding comprises withholding food from said subject for at least six hours prior to oral administration.

23. The method of claim 18, wherein said interferon-τ is ovine or bovine interferon-τ.

24. The method of claim 23, wherein said interferon-τ has an amino acid sequence corresponding to the sequence presented as SEQ ID NO:2.

25. The method of claim 23, wherein said orally administering includes orally administering interferon-τ as a solid dosage form.

26. The method of claim 23, wherein said orally administering includes orally administering interferon-τ as a liquid dosage form.

27. The method of claim 23, wherein said orally administering is at a dose of at least about 1×10⁴ Units/day.

28. The method of claim 23, wherein said administering is effective to treat an autoimmune condition.


SEQUENCE LISTING (3 sequences)

SEQ ID NO: 1 - DNA, 516 bp, Artificial Sequence
  synthetic gene encoding ovine interferon-tau
tgctacctgtcgcgaaaactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaggttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

SEQ ID NO: 2 - protein, 172 aa, Artificial Sequence
  amino acid encoded by SEQ ID NO1
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 172 aa, Ovis aries; identical to SEQ ID NO: 2